(19) United States
(12) Patent Application Publication
(10) Pub. No.: US 2016/0174812 A1
(43) Pub. Date: Jun. 23, 2016

(54) Multi-Spot Laser Probe With Sapphire Ball And Molded Glass

(71) Applicant: Novartis AG, Basel (CH)

(72) Inventors: Alexander N. Artsyukhovich, Lake Forest, CA (US); Tammo Heeren, Aliso Viejo, CA (US); Michael J. Papac, North Tustin, CA (US); Ronald T. Smith, Irvine, CA (US); Dustin J. Bouch, Newport Beach, CA (US)

(21) Appl. No.: 14/576,703

(22) Filed: Dec. 19, 2014

(51) Int. Cl. A61B 1/00

(52) U.S. Cl. CPC A61B 1/0011; A61B 1/00064

(57) ABSTRACT

A method of making an optical surgical probe includes providing and heating a substrate; melting a piece of glass on the heated substrate; immersing a ball lens into the molten piece of glass; heating a molding pin having a concave prism end; wetting the concave prism end of the molding pin with a molten glass drop; inserting the molding pin into the molten glass aligned with the ball lens; sliding a cannula along an axis of the molding pin toward the substrate to create a cannula/glass-optical-element/ball lens probe-assembly; cooling the probe-assembly; and extracting the probe-assembly.

### BACKGROUND

1. Technical Field

This invention relates to an optical surgical probe and, more particularly, to a multi-spot laser surgical probe using a faceted optical element.

2. Related Art

Optical surgical probes deliver light to a surgical region for a variety of applications. In some applications, it may be useful to deliver light to multiple spots in the surgical region. For example, in pan-retinal photocoagulation of retinal tissue, an optical surgical probe that is configured to split a single laser or light beam into multiple beams focused to multiple retinal spots can cause photocoagulation at these spots simultaneously. Photocoagulating at n=2, 4, 6 spots simultaneously can reduce the time of the pan-retinal photocoagulation procedure approximately by a factor of 2, 4, or 6.

Various probe designs have been employed to produce multiple beams for a multi-spot pattern. For example, some probes include a diffractive element to divide a single beam into multiple beams corresponding to higher diffractive orders. Such diffractive elements are typically positioned inside the surgical probe, as positioning them at the end of the probe would pose substantial design challenges. However, positioning the diffractive elements away from the end of the probe can limit their functionalities.

Further, it has proven difficult to produce only the desired diffractive orders with high intensity while keeping the intensity of undesired diffractive orders sufficiently low. In addition, the performance of diffractive elements in different refractive media can differ substantially.

Another general challenge for the design of beam splitting probes is to fit them into a sufficiently small cannula at the end of the probe. The leading probes today have 23 Gauge cannulas, i.e. an outer diameter of about 0.650 mm. It is a non-obvious challenge to design the beam splitting elements to fit into these very narrow cannulas. Thus, a need persists for an optical surgical probe that can produce well-controlled multiple spots at a surgical target region using optical elements that can fit into the narrow cannula at the probe's end.

### SUMMARY

In particular embodiments of the present invention, a method of making an optical surgical probe can comprise: providing and heating a substrate; melting a piece of glass on the heated substrate; immersing a ball lens into the molten piece of glass; heating a molding pin having a concave prism end; wetting the concave prism end of the molding pin with a molten glass drop; inserting the molding pin into the molten glass aligned with the ball lens; sliding a cannula along an axis of the molding pin toward the substrate to create a cannula/glass-optical-element/ball lens probe-assembly; cooling the probe-assembly; and extracting the probe-assembly.

In particular embodiments of the present invention, a method of making an optical surgical probe can comprise: providing and heating a substrate; melting a piece of glass on the heated substrate; heating a molding pin having a concave prism end that is a negative of a faceted proximal surface of a glass optical element with facets curved to focus beam-components to multiple spots in an image plane; wetting the concave prism end of the molding pin with a molten glass drop; inserting the molding pin into the molten glass; sliding a cannula along an axis of the molding pin toward the substrate to create a cannula/glass-optical-element probe-assembly; cooling the probe-assembly; and extracting the probe-assembly.

Other objects, features and advantages of the present invention will become apparent with reference to the drawings, and the following description of the drawings and claims.

### BRIEF DESCRIPTION OF THE DRAWINGS

FIG. 1 illustrates an optical surgical probe

FIG. 2 illustrates a perspective view of an optical surgical probe.

FIG. 3 illustrates an embodiment of an optical surgical probe without a ball lens.

FIG. 4 illustrates a flow chart of a method of making an optical surgical probe with a ball lens.

FIGS. 5A-F illustrate steps of making an optical surgical probe.

FIG. 6 illustrates a flow chart of a method of making an optical surgical probe without a ball lens.

### DETAILED DESCRIPTION

Some surgical probes split an incoming light beam, or laser beam, with a multi-spot generator that employs a faceted optical element to split the beam. The multi-spot generator can be positioned at the end of the cannula of the probe. It can also include a ball lens to focus the split beams. For ease of manufacture, the faceted optical element is typically made of an adhesive, which is cured after having been formed to the desired shape.

A problem with such probe designs is that for a variety of reasons the multi-spot generator can heat up when the beam is applied during the surgical procedure. In fact, a local operating temperature Tₒₚ of the multi-spot generator can rise close to or even beyond a critical temperature T_{c} of the cured adhesive: Tₒₚ>T_{c}. Here, the critical temperature T_{c} can reflect different undesirable physical processes that can have a negative or even critical effect on the performance of the probe. In some designs and applications, T_{c} can represent T_{c1}, the temperature when the cured adhesive gets detached from the cannula. In other cases, T_{c} can represent T_{c2}, the critical temperature where the cured adhesive becomes soft and malleable to a critical degree. T_{c2} is sometimes also called the softening temperature, or anneal temperature. Finally, in some cases, the critical temperature T_{c} can represent T_{c3}, the melting temperature of the cured adhesive. As is well known in the glass science and industry, the values of T_{c1}-T_{c3} depend to some degree on the details of their measurement and definition, so these temperatures are defined with a few percent tolerance. Any one of these physical processes can lead to the cured adhesive becoming soft or detached to a degree that the performance of the multi-spot generator deteriorates or even gets critically compromised.

In some cases, the performance deterioration can be gradual, such as the facets gradually change their shape thus causing the spots to form farther and farther from their intended location as the surgery proceeds. In other cases, the performance deterioration can be abrupt and critical, such as the ball lens getting loose and falling out at the end of the cannula into the eye chamber itself. Both the gradual and especially the critical performance deterioration are associated with the use of cured adhesives in these probes. Therefore, there is a need for optical surgical probes with multi-spot generators, whose faceted optical elements have critical temperatures T_{c} that are higher than the local operating temperature Tₒₚ of the probe. Such probes should not exhibit the just-described types of performance deterioration.

Recognizing this problem and positing a path to its solution has not been obvious, as it involved noticing that a systematic malfunction keeps recurring in the probes, properly identifying the cause of the malfunction among the numerous possibilities as the softening or melting of the cured adhesive, and discovering that materials and manufacturing processes with high enough T_{c} can be developed that solve the problem. Switching to a high T_{c} material is not a design choice either, because the manufacturing and manipulating high T_{c} materials is substantially more challenging than low T_{c} materials. Therefore, without having discovered the problem and that the path to its solution requires a high T_{c} material, a designer would have rather chosen a low T_{c} material instead.

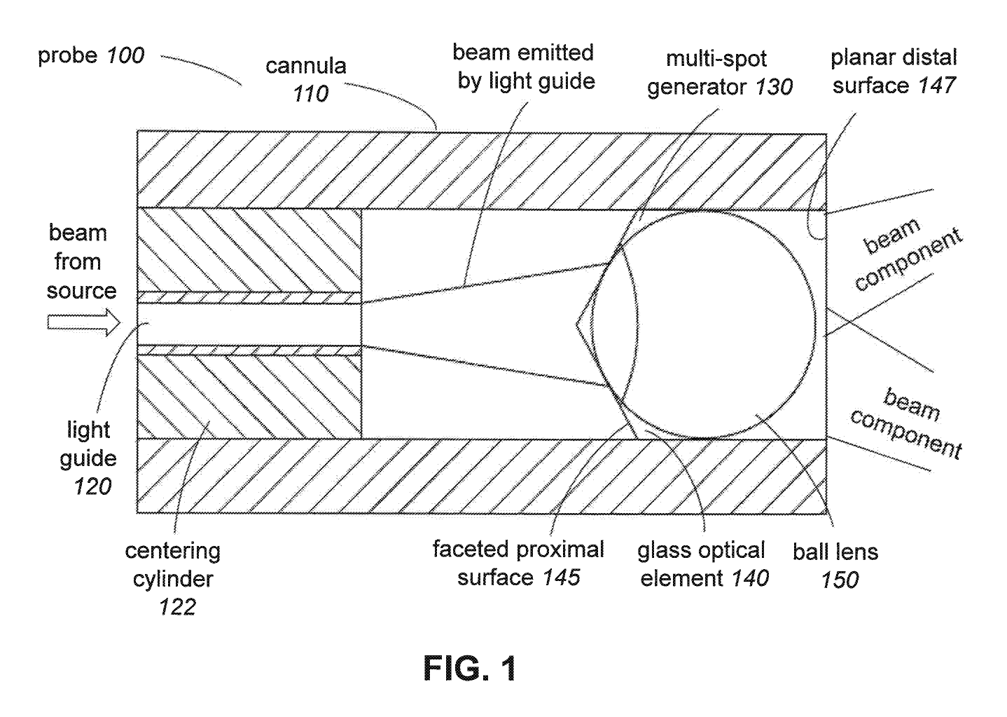

FIG. 1 illustrates embodiments that offer solutions for the above problems. An embodiment of an optical surgical probe 100 can include a cylindrical cannula 110 and a light guide 120 that is partially positioned within the cannula 110. The light guide 120 can be centered by a centering cylinder 122. The light guide 120 can be configured to receive a light beam from a light source through a proximal end, and to emit the light beam through a distal end. The light beam can be generated by a traditional incoherent light source, a light emitting diode (LED), or any coherent light source, such as a laser source. The wavelength of the light beam can take a wide variety of values. Direct tracking of the surgical procedure can be achieved for the surgeon by utilizing a light beam with a wavelength in the visible spectrum approximately in the 400-700 nm range. In some specific cases, the wavelength can be in the 500-600 nm range, such as 532 nm.

The light guide 120 can be an optical fiber, such as a glass fiber. The optical fiber can have a core-cladding-jacket structure. The light beam can be emitted by the light guide 120 with some degree of divergence. The divergent emitted light beam can have, for example, a numerical aperture NA in the range of 0.00-0.30, in some cases in the range of 0.10-0.20.

The light guide 120 can emit the light beam towards a multi-spot generator 130 that is positioned at a distal end of the cannula 110. The multi-spot generator 130 can be positioned at the very end of the cannula 110, its end flush with the cannula's end. In other embodiments, the multi-spot generator can be set back somewhat from the very end of the cannula 110.

Some embodiments of the multi-spot generator 130 can include an optical element 140 that is made of a high T_{c} critical temperature material, such as glass. The glass optical element can have a faceted proximal surface 145. The faceted proximal surface 145 can be configured to receive the light beam from the light guide 120, and to split, or to refract the light beam into beam-components. In various embodiments, the faceted proximal surface 145 can have 2, 4, 6 or other suitable number of facets. In some embodiments, the facets can be planar surfaces oblique to the optical axis of the cannula 110. The facets can make an angle with the optical axis in the 10-50 degrees range, in some cases in the 20-40 degrees range. In some embodiments, the facets can be non-planar, curved surfaces.

The multi-spot generator 130 can further include a ball lens 150 inside the glass optical element 140. The ball lens 150 can be fully inside the glass optical element 140. The ball lens 150 can be close to the faceted proximal surface 145, or to a planar distal surface 147 of the glass optical element 140, but can avoid touching or interrupting these surfaces to avoid disturbing the wavefronts. The ball lens 150 can be configured to focus the beam-components to multiple spots in an image plane. The focused beam components can be emitted through the distal surface 147 of the glass optical element 140, as shown. In an image plane, located at 1-8 mm from the distal end of the cannula 110, such as at 4 mm, the beam components can form spots, spaced apart by 0.5-2 mm. Embodiments of the faceted proximal surface 145 with 4 facets create 4 beam components that create 4 spots, for example arranged in a square. A suitably large surgical area can be covered or processed efficiently by placing these spot-squares in a repeating pattern.

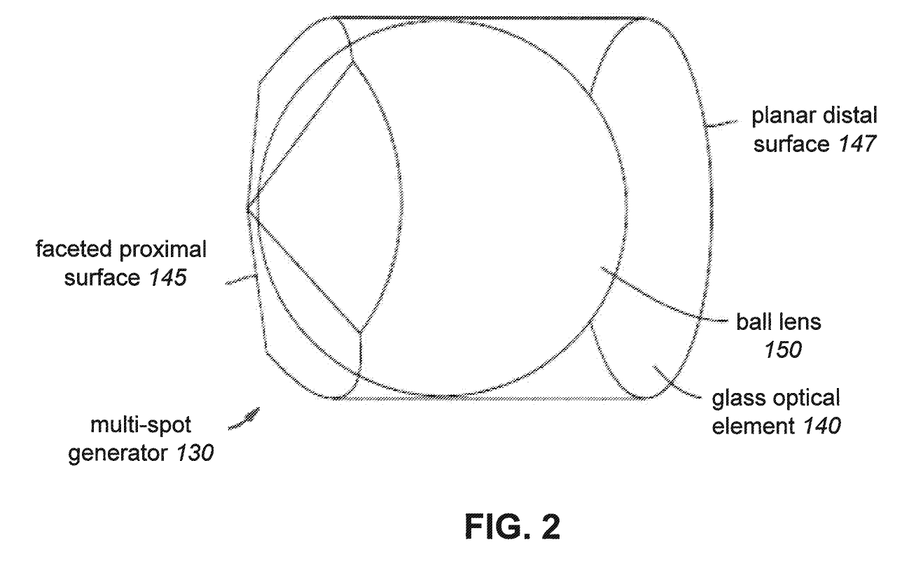

FIG. 2 illustrates an embodiment of the multi-spot generator 130 from a perspective view. As in FIG. 1, this embodiment of the multi-spot generator 130 can include the glass optical element 140 with the faceted proximal surface 145 and the planar distal surface 147. The glass optical element 140 can further include the ball lens 150.

The described embodiments of the optical surgical probe 100 address the above described problems by forming the optical element 140 from glass. As it is known, while various cured adhesives typically have critical temperatures T_{c} in the few hundred centigrade range, such as T_{c}(adhesive)˜200-300 C (centigrade), large classes of glass have critical temperatures in the T_{c}(glass)˜500-1,000 C range. At the same time, the local operating temperature Tₒₚ of surgical probes is often in the 100-300 C range. Therefore, optical surgical probes 100 that include an optical element 140 that is made of glass instead of a cured adhesive are capable of operating at a local operating temperature Tₒₚ that is lower than a critical temperature T_{c} of the optical element with a sufficient margin to avoid the described undesirable performance deterioration. Here, Tₒₚ refers to a local operating temperature of the glass optical element 140 itself, as there can be substantial temperature gradients throughout the surgical optical probe 100, and the critical temperature T_{c} is one of a temperature T_{c1} where the glass optical element 140 detaches from the cannula 110, a temperature T_{c2} where the glass optical element 140 becomes soft to a critical degree, and a melting temperature T_{c3} of the glass optical element 140.

In some embodiments, a critical temperature T_{c} of the glass optical element 140 is higher than 500 centigrade: T_{c}>500 C. Examples of such glass optical elements 140 include molded glass.

Concerning the optical characteristics of the glass of the optical element 140, an index of refraction of the glass optical element 140 can be between 1.4 and 1.6 in some embodiments.

In some embodiments of the optical surgical probe 100 the cannula 110 can be sized to be 23 Gauge or smaller. This translates to the cannula 110 having an outer diameter less than 700 microns and an inner diameter less than 400 microns. In some embodiments, the cannula has an outer diameter of 650 microns plus minus 10 microns.

In some embodiments, a diameter of the ball lens 150 can be between 100 and 500 microns. In some embodiments, the diameter of the ball lens 150 can be between 350 and 400 microns.

In some embodiments, the ball lens 150 can comprise sapphire. An index of refraction of the ball lens can be in the 1.55-1.85 range, in some embodiments in the 1.7-1.8 range.

In some embodiments of the probe 100, the glass optical element 150 comprises molded glass.

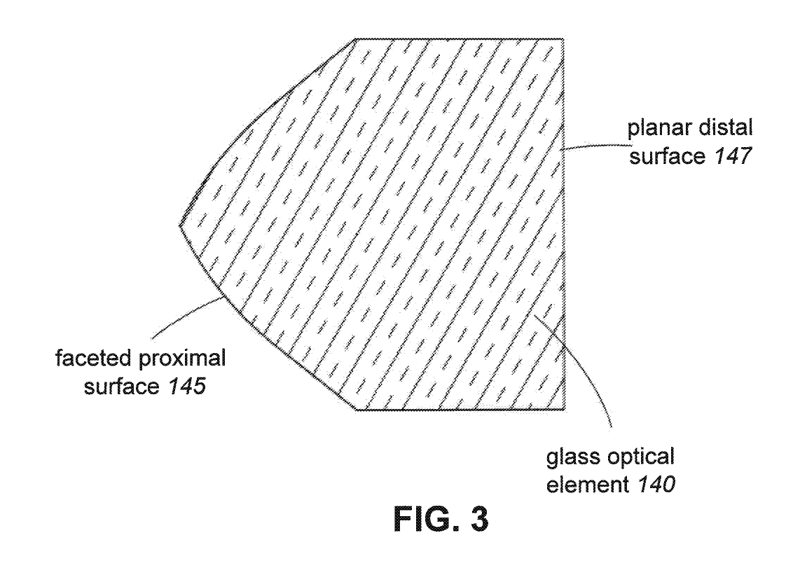

FIG. 3 illustrates that in some embodiments, the glass optical element may not include a ball lens 150. In such embodiments, the glass optical element 140 can still include a faceted proximal surface 145. In these embodiments, the facets may be curved. Curved facets can not only split the incident beam into beam components, but also focus the beam components to spots in an image plane.

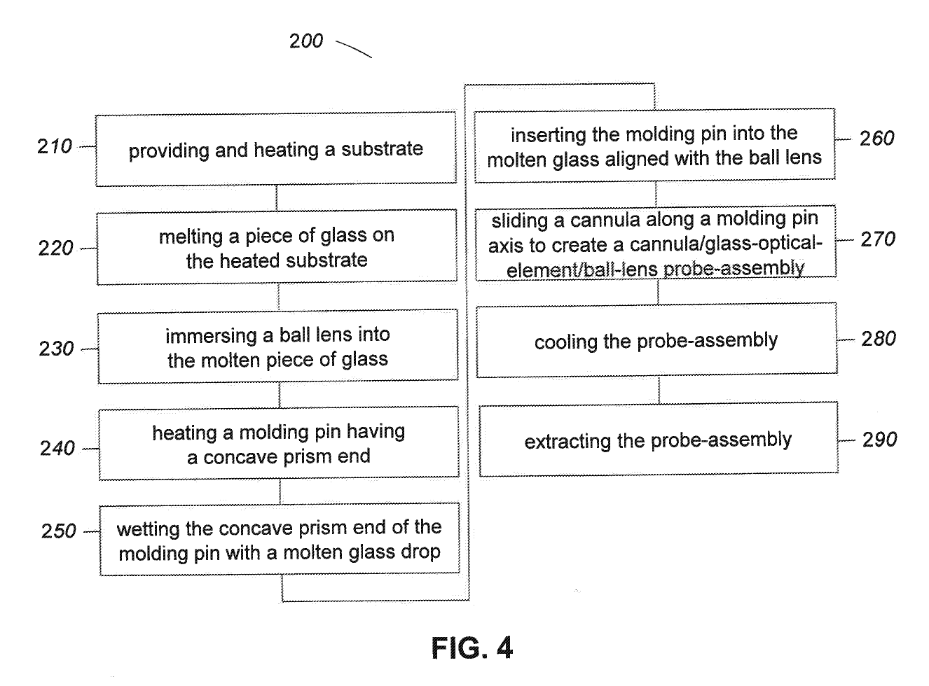

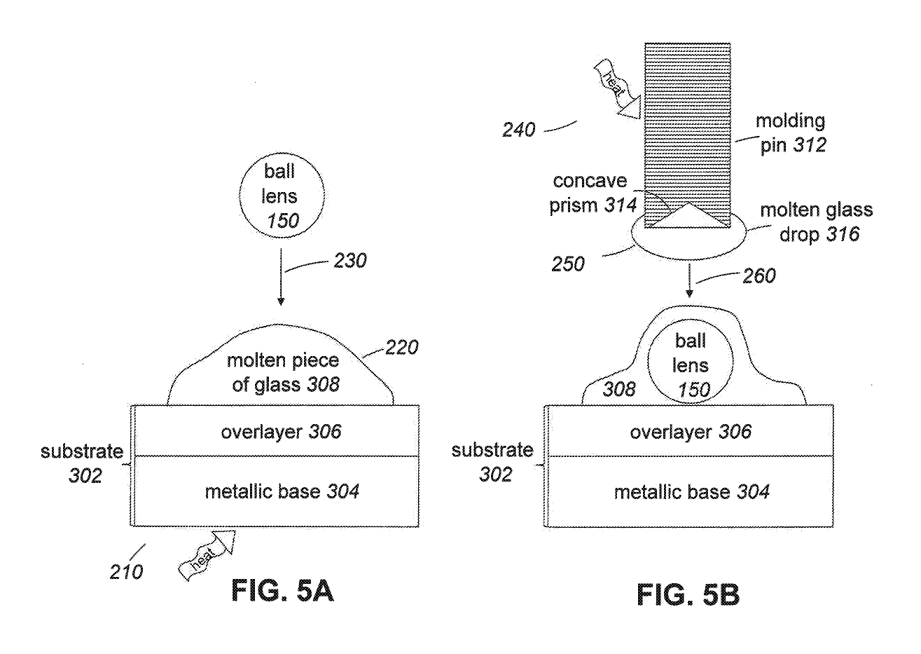

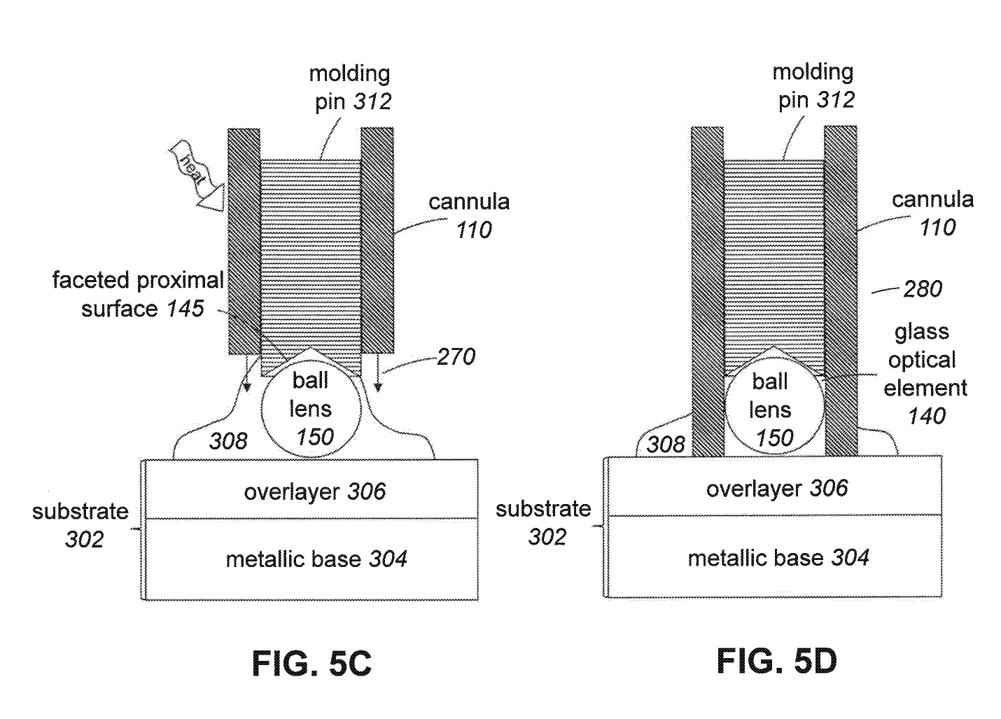

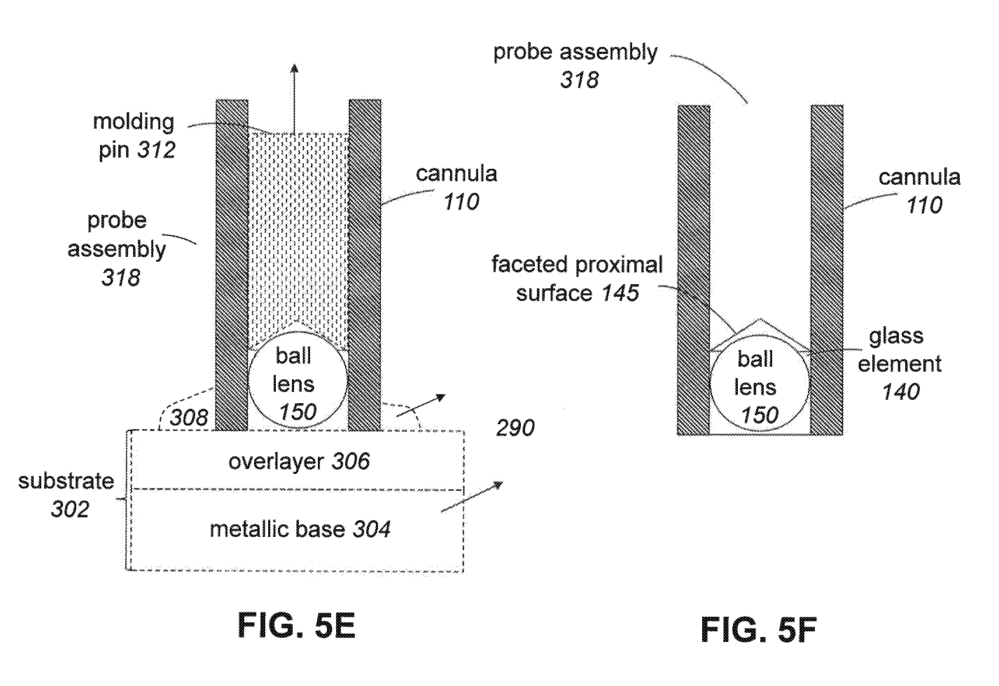

FIG. 4 is a flow chart illustrating an example method 200 for forming an optical surgical probe 100 with a glass optical element 140. FIGS. 5A-F illustrate some of the steps of method 200.

The method 200 can include providing and heating a substrate 210; melting a piece of glass on the heated substrate 220; immersing a ball lens into the molten piece of glass 230; heating a molding pin having a concave prism end 240; wetting the concave prism end of the molding pin with a molten glass drop 250; inserting the molding pin into the molten glass aligned with the ball lens 260; sliding a cannula along an axis of the molding pin toward substrate to create a cannula/glass-optical-element/ball-lens probe-assembly 270; cooling the probe-assembly 280; and extracting the probe-assembly 290.

FIG. 5A illustrates that in some embodiments the providing 210 can include providing a substrate 302 that is a composite substrate 302. In some cases the composite substrate 302 can include a metallic base 304 and an overlayer 306. The metallic base 304 can be formed from a wide variety of metals, including copper. A general property of metals is that they have high heat conductivity and thus when heated, their temperature rises evenly across the entire metallic base 304.

The overlayer 306 can include ruby, sapphire, fused silica or other materials with similar thermodynamic characteristics. These characteristics include thermal expansion and conduction characteristics. The overlayer 306 can be sufficiently extended to prevent a direct contact between the piece of glass 308 and the metallic base 304. A direct contact between the piece of glass 308 and the metallic base 304 could introduce substantial mechanical and crystalline strains into the piece of glass 308 with the fabricating temperature changes because of the substantially different thermal expansion properties of glasses and metals. Such strains can introduce optical disturbances and even mechanical breakdown of the piece of glass 308, such as cracking In addition, molecules from the metallic base 304 or its oxidized metal oxide surface may diffuse into the bottom surface of the piece of glass 308, causing degradation of its performance including increased heating from increased absorbance of light while the multi-spot optical surgical probe 100 is being used.

The providing 210 also includes heating the substrate 302 with, for example, an electric heater, a radiation heater, a laser beam, or another known heating system (indicated by the wavy heat-radiation sign). In embodiments where a laser beam is used, the laser beam can be directed to the metallic base 304, for example to its side opposite to the overlayer 306. The metallic base 304 in response heats up evenly because of its high thermal conductivity.

In some embodiments, the melting 220 can include providing the piece of glass 308 with a critical temperature T_{c} greater than a local operating temperature Tₒₚ of the glass optical element 140 of the optical surgical probe 100, with reference to FIG. 1. As before, the critical temperature T_{c} can be one of a temperature T_{c1}, where the glass optical element 140 detaches from the cannula 110, a temperature T_{c2}, where the glass optical element 140 becomes soft to a critical degree, and a melting temperature T_{c3} of the glass optical element 140. The local operating temperature Tₒₚ can be a temperature of the glass optical element 140 during an operation of the optical surgical probe 100.

As explained above, embodiments of probe 100 that have their optical element 140 formed from glass can be operated at a local operating temperature Tₒₚ lower than the critical temperature T_{c} of the glass optical element 140 because the critical temperature T_{c} of glass is higher than that of cured adhesives. For example, in some embodiments, the piece of glass 308 can have a critical temperature greater than 500 C. Accordingly, in some embodiments, the piece of glass 308 can include molded glass.

During the immersing 230, it can be useful to ensure that the ball lens 150 does not roll off the substrate 302, and is positioned preferably in a central or symmetrical position in the molten piece of glass 308 on the substrate 302.

FIG. 5B illustrates that the heating 240 can include providing a molding pin 312 with a concave prism end 314 that can be a negative of a faceted proximal surface 145 of the glass optical element 140 of the optical surgical probe 100. As described above in detail, the faceted proximal surface can have 2, 4, 6 facets, oblique to an axis of the probe 100, thus configured to split an incoming axial beam into beam components.

The heating 240 can also include heating the molding pin 312. If the molding pin 312 is inserted into the hot molten piece of glass 308 in a cold state, then the two temperatures will equilibrate. This equilibrated temperature may be below the softening temperature of the piece of glass 308, creating an essentially solidified glass region in front of the molding pin 312, strongly obstructing, possibly even blocking its further movement to its intended depth within the piece of glass 308. If the equilibrated temperature is above the softening temperature, but still below the melting temperature, then stresses will be induced between the molding pin 312 and portions of the piece of glass 308, as well as within the piece of glass 308. These stresses typically do not relax but freeze in place when the piece of glass 308 eventually solidifies, leading to the possibility of reduced reliability, including stress-induced cracking within the glass, as well as below-specification device performance.

Pre-heating the molding pin 312 to a suitable temperature as part of the heating 240 can avoid the formation of such a blocking solidified glass region and the inducing of stressed glass regions in the piece of glass 308. In some embodiments, the suitable temperature can be a temperature that is greater than 50% of the melting temperature T_{c3} of the piece of glass 308. As before, the heating 240 of the molding pin 312 can be performed with an electric heater, a radiation heater, a laser beam, or another known heating system, as shown.

The wetting 250 of the concave prism end 314 at a distal end of the molding pin 312 with a molten glass drop 316 can make it easier to move or insert the molding pin 312 into the molten piece of glass 308, as at the moment of contact the molten glass of the drop 316 meets the molten glass 308, ideally at a single point, and the two molten glasses adapt easier than the molding pin 312 to the molten glass 308. Further, the wetting 250 by the molten glass drop 316 allows the molding pin 312 to equilibrate with a molten glass prior to the inserting 260, thus the inserting 260 into the molten piece of glass 308 will not cause or require an additional thermal expansion of the molding pin 312 and the concomitant strains in the molten piece of glass 308. These considerations make the wetting 250 another element of the method 200 that enables the manipulation and control of the high melting temperature glass instead of the low melting temperature adhesives to form the multi-spot generator 130.

The inserting 260 can include inserting the molding pin 312 into the molten piece of glass 308 centrally aligned with the ball lens 150. This step can be an aspect of aligning a center and axis of the faceted proximal surface 145, formed by the concave prism end 314 from the molten piece of glass 308 with a center and axis of the ball lens 150.

The inserting 260 can also include stopping the inserting of the molding pin 312 before the molding pin 312 makes contact with the ball lens 150. This step can be part of the process that forms the faceted proximal surface 145 without interruption or deformation by the ball lens 150.

FIG. 5C illustrates that in some embodiments the sliding 270 can include heating the cannula 110 prior to the sliding. The heating is again indicated by the wavy radiation sign and can be performed by an electric heater, a radiation heater or a laser beam, among others. As before, this heating step can be a further aspect of reducing and minimizing the thermal expansion of the cannula 110 once in contact with the molten piece of glass 308, thus reducing and minimizing the mechanical strain induced in the molten piece of glass 308. Additionally, upon the subsequent cooling the heated cannula 110 after the sliding 270 is completed, its inner diameter shrinks, thus making the connection between the molten piece of glass 308 tighter and more reliable.

The sliding 270 can also include sliding the cannula 110 until it makes contact with the substrate 302, in some embodiments with the overlayer 306. Once the cannula 110 makes contact with the substrate 302, the molten piece of glass 308 is essentially transformed into the glass optical element 140 by the cannula 110 and by the molding pin 312. With this, the cannula 110, the glass-optical-element 140 and the ball lens 150 can form a probe-assembly 318 as best illustrated in FIG. 5F.

Once the probe-assembly 318 is formed, the cooling 280 lowers the temperature of the probe-assembly 318 slowly and gradually. A slow and gradual cooling can minimize the introduction of mechanical strains into the glass optical element 140, thus maintaining the high optical quality of the just-formed glass optical element 140, whereas a fast cooling could induce strains and lower the optical quality of the glass optical element 140.

Finally, FIG. 5E illustrates that the extracting 290 can include removing the molding pin 312 from the cannula 110, e.g. by withdrawing the molding pin 312 from the cannula 110, indicated by dotted line and arrow. The extracting 290 can further include removing the probe-assembly 318 from the substrate 302, e.g. by breaking off or peeling off the probe-assembly 318 from the substrate 302. Finally, the extracting 290 can also include removing excess glass from the probe-assembly 318, e.g. the irregular glass droplets and shards that remained from the portion of the molten piece of glass 308 pushed away by the molding pin 312 and the cannula 110.

The extracting 290 of the probe-assembly 318 separates and completes the fabrication of the probe-assembly 318, as shown in FIG. 5F.

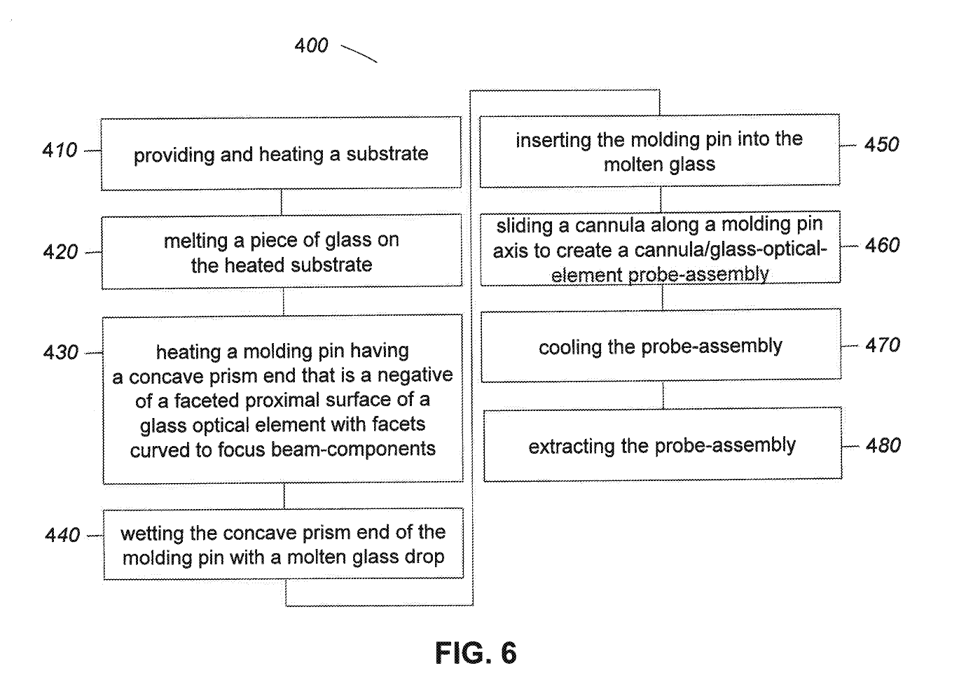

FIG. 6 illustrates a method 400 of making a ball-lens-free optical surgical probe of the type shown in FIG. 3. The method 400 can include providing 410 and heating a substrate; melting 420 a piece of glass on the heated substrate; heating 430 a molding pin having a concave prism end that is a negative of a faceted proximal surface of a glass optical element with facets curved to focus beam-components to multiple spots in an image plane; wetting 440 the concave prism end of the molding pin with a molten glass drop; inserting 450 the molding pin into the molten glass; sliding 460 a cannula along an axis of the molding pin toward substrate to create a cannula/glass-optical-element probe-assembly; cooling 470 the probe-assembly; and extracting 480 the probe-assembly.

Most steps of method 400 are analogous to those of method 200. The differences, e.g. the absence of an immersing of the ball lens 150, are motivated by the fact that the curving of the facets of the faceted proximal surface 145 can perform a role analogous to that of the ball lens 150 itself: the focusing of the beam-components generated by the faceted proximal surface 145 onto multiple spots in an image plane. Therefore, it is not necessary to immerse a ball lens 150 into the molten glass.

The present invention is illustrated herein by example, and various modifications may be made by a person of ordinary skill in the art. Although the present invention is described in detail, it should be understood that various changes, substitutions and alterations can be made hereto without departing from the scope of the invention as claimed.

1. A method of making an optical surgical probe, the method comprising:
providing and heating a substrate;
melting a piece of glass on the heated substrate;
immersing a ball lens into the molten piece of glass;
heating a molding pin having a concave prism end;
wetting the concave prism end of the molding pin with a molten glass drop;
inserting the molding pin into the molten glass aligned with the ball lens;
sliding a cannula along an axis of the molding pin toward the substrate to create a cannula/glass-optical-element/ball lens probe-assembly;
cooling the probe-assembly; and
extracting the probe-assembly.

2. The method of claim 1, the providing comprising:
providing a composite substrate, comprising
a metallic base, and
an overlayer, comprising at least one of ruby, sapphire and fused silica.

3. The method of claim 2, the providing comprising:
providing an overlayer that is configured to prevent a direct contact between the piece of glass and the metallic base.

4. The method of claim 1, the providing comprising:
heating the substrate with at least one of an electric heater, a radiation heater, and a laser beam.

5. The method of claim 1, the melting comprising:
providing a piece of glass with a critical temperature greater than a local operating temperature of the glass optical element of the optical surgical probe.

6. The method of claim 5, wherein:
the critical temperature is one of a temperature where the glass optical element detaches from the cannula, a temperature where the glass optical element becomes soft to a critical degree, and a melting temperature of the glass optical element.

7. The method of claim 1, the melting comprising:
providing a piece of glass with a critical temperature greater than 500 C.

8. The method of claim 1, comprising:
providing a molded glass as the piece of glass.

9. The method of claim 1, the heating comprising:
providing a molding pin with a concave prism end that is a negative of a faceted proximal surface of the glass optical element of the optical surgical probe.

10. The method of claim 1, the heating comprising:
heating the molding pin to a temperature that is greater than 50% of the melting temperature of the piece of glass.

11. The method of claim 1, the inserting comprising:
inserting the molding pin into the molten glass centrally aligned with the ball lens; and
stopping the inserting before the molding pin makes contact with the ball lens.

12. The method of claim 1, the sliding comprising:
heating the cannula prior to the sliding.

13. The method of claim 1, the sliding comprising:
sliding the cannula until it makes contact with the substrate.

14. The method of claim 1, the extracting comprising:
removing the molding pin;
removing the probe-assembly from the substrate; and
removing excess glass from the probe-assembly.

15. A method of making an optical surgical probe, the method comprising:
providing and heating a substrate;
melting a piece of glass on the heated substrate;
heating a molding pin having a concave prism end that is a negative of a faceted proximal surface of a glass optical element with facets curved to focus beam-components to multiple spots in an image plane;
wetting the concave prism end of the molding pin with a molten glass drop;
inserting the molding pin into the molten glass;
sliding a cannula along an axis of the molding pin toward the substrate to create a cannula/glass-optical-element probe-assembly;
cooling the probe-assembly; and
extracting the probe-assembly.
